(19) United States
(12) Patent Application Publication
(10) Pub. No.: US 2008/0114439 A1
(43) Pub. Date: May 15, 2008

(54) Non-occluding dilation device

(76) Inventors: Venkatesh Ramaiah, Scottsdale, AZ (US); Robert McNutt, Mesa, AZ (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 11/977,415

(22) Filed: Oct. 23, 2007

Related U.S. Application Data
(63) Continuation-in-part of application No. 11/820,726, filed on Jun. 19, 2007.
Application No. 11/820,726 is a continuation-in-part of application No. 11/478,340, filed on Jun. 28, 2006.
(60) Provisional application No. 60/595,378, filed on Jun. 28, 2005.

(51) Int. Cl. A61F 2/90; A61F 2/84

(52) U.S. Cl. 623/1.11; 623/1.15

(57) ABSTRACT

An assembly including a device for dilating a vessel or a structure (such as a stent or stent graft) within a vessel wherein the device comprises a plurality of wires that are spaced apart when the device is dilated so as to allow fluid to flow through the device. Thus, when in use the device does not occlude or substantially hinder the flow of blood through a vessel or into side vessels. The device may also be flexible enough to be easily maneuvered through tight bends in a vessel and/or be able to conform to varying diameters within a single vessel or within multiple vessels.

### CROSS REFERENCE TO RELATED APPLICATIONS

This application is a continuation-in-part of and claims priority to (1) U.S. Utility application Ser. No. 11/820,726, filed Jun. 19, 2007 (FIGS. 8 through 26 of which are incorporated herein by reference), which is a continuation-in-part of U.S. Utility application Ser. No. 11/478,340, filed Jun. 28, 2006, which claims the benefit of Provisional Application No. 60/595,378, filed Jun. 28, 2005, and (2) to U.S. Utility application Ser. No. 11/478,340 filed Jun. 28, 2006.

### FIELD OF THE INVENTION

The present invention relates generally to medical devices, and more particularly to a medical device for the dilation of blood vessels and/or the dilation of structures positioned within blood vessels.

### BACKGROUND OF THE INVENTION

Conventional systems for dilating blood vessels and/or structures (e.g., stents or stent grafts) positioned in a blood vessel utilize balloon-like structures (“balloon dilators”). Such structures are typically made from essentially impermeable materials. When such a device is expanded to perform a dilation, blood flow is entirely or substantially occluded through the blood vessel in which the balloon dilator is being used. Such an occlusion of blood flow could, if continued for too long, harm the patient, since portions of the body downstream of the balloon dilator will not receive blood while the flow is occluded or substantially hindered. Thus, the length of time balloon dilators may be dilated is limited and this can hinder proper completion of the dilation procedure.

A similar problem with balloon dilators arises when a dilation procedure is being performed in a portion of the circulatory system where there is a branch in the blood vessels, such as where the iliac or renal arteries are side vessels that branch from the aorta. In that case a balloon dilator may cover a side vessel and partially or totally occlude blood flow to the side vessel.

Another problem with balloon-like dilators is called the “windsock effect.” Because blood flow is substantially or entirely occluded when balloon dilators are dilated, the blood pressure upstream of the balloon dilator can be significant and may cause the balloon dilator, and any structure (such as a stent or stent graft) positioned in the blood vessel and that is being dilated by the balloon dilator, to move out of the desired position, effectively pushed down stream (i.e., in the antegrade direction) by the upstream blood pressure. Because of this problem accurate placement of such structures can be difficult utilizing balloon dilators.

### DEFINITIONS

As used herein, in addition to the other terms defined in this disclosure, the following terms shall have the following meanings:

“Assembly” means a device according to the invention assembled as part of or connected to a catheter so that it can be advanced into a vessel.

“Collapsed” when referring to a device according to the invention means that the device is in its relaxed, undilated position. The device would normally be in its collapsed position when introduced into a vessel and/or when retained within a cover sheath of a triaxial catheter.

“Contraction” of a device or “contracting” a device means that its diameter is being or has been reduced from a dilated position.

“Criss-cross” pattern means a wire pattern wherein the wires cross one another as shown, for example, in FIGS. 13-20.

“Device” or “dilation device” means a structure for (a) dilating a vessel, and/or (b) dilating a structure inside of one or more vessels (such as an endograft stent or stent graft) to be deployed or repositioned within one or more vessels.

“Diameter” as used in connection with a vessel means the approximate diameter of a vessel since vessels are seldom perfectly cylindrical. “Diameter” as used with respect to any man-made structure means the approximate diameter.

“Diameter disparity ratio” means the disparity of the diameter of a single vessel. Vessels, particularly diseased vessels, may not have a relatively constant diameter and the diameter can suddenly increase or decrease. For example, the diameter of a vessel may suddenly change from an initial diameter to a diameter of 1.5 times the initial diameter, in which case the diameter disparity ratio would be 1.5:1. A diameter disparity ratio or multi-vessel diameter disparity ratio (as defined below) to which a device according to some aspects of the invention could conform is one or more of the ratios between 1.2:1 and 3.4:1, including diameter disparity ratios of 1.2:1, 1.4:1, 1.6:1, 1.8:1, 2.0:1, 2.2:1, 2.4:1, 2.6:1 and 2.8:1, 3.0:1 and 3.4:1.

“Dilated” refers to a device according to the invention when it is expanded. A device dilated within a vessel may be dilated for the purpose of dilating the vessel itself and/or for dilating a structure within the vessel. “Expanded” and “dilated” have the same meaning when used in connection with a device according to the invention.

“Fluid” means any bodily fluid, such as blood.

“Fully dilated” or “fully expanded” means the maximum amount a device according to the invention can be dilated (as measured at its greatest diameter) when unhindered by external structures (such as a vessel) and when dilated using the delivery system of a catheter to which the device is attached.

“Kink radius” refers to the radius to which a device according to the invention can be formed without the device permanently deforming (i.e., without “kinking”). If the device is mounted on a catheter the kink radius refers to the kink radius of the entire assembly, i.e., the device mounted to a biaxial or triaxial catheter (with the sheath covering the device), since the entire assembly moves through the vessel when the device is advanced into place. The lower the kink radius the greater the resistance of the device or assembly to kinking. FIGS. 21-23 show measurement of the kink radius with respect to an embodiment of the present invention. “Kink radius” and methods for testing same are discussed in “Pigtail Catheters Used for Percutaneous Fluid Drainage Comparison of Performance Characteristics,” Douglas B. Macha, John Thomas and Rendon C. Nelson, Radiology vol. 238: Number 3 (March 2006), the contents of which that are related to kink testing are incorporated herein by reference.

“Multi-vessel diameter disparity ratio” means the disparity of the diameters of two vessels. When a device according to the invention is used it may be deployed and dilated within two vessels simultaneously and the two vessels may have different, respective diameters. For example, if one vessel has a first diameter and the second vessel has a second diameter 1.8 times as large as the first diameter, the multi-vessel diameter disparity ratio would be 1.8:1. A device according to some aspects of the invention could conform to one or more of the multi-vessel diameter ratios between 1.2:1 and 3.4:1.

“Pressure drop” means the reduction in pressure in part of a vessel when a device is (a) dilated within the vessel, or (b) dilated in another vessel but totally or partially covering the opening to the vessel (in which case the vessel may be referred to as a “side vessel”). When a balloon dilator is fully dilated within a vessel the pressure upstream of the balloon dilator increases significantly while the pressure downstream of the balloon dilator, or in a side vessel covered by the balloon dilator, can reach substantially zero (meaning that the balloon dilator has blocked most or all of the blood flow). As an example, if the pressure at a location in a vessel is 100 mm Hg (i.e., a pressure of 100 millimeters of mercury) before a device is dilated within the vessel, and the pressure at the same location in the vessel is 10 mm HG after the device is dilated, the pressure drop would be 90%, i.e., 100−10=90, and 90/100=90%. Similarly, for the same vessel if the pressure after dilation were 20 mm Hg the pressure drop would be 80%, if the pressure after dilation were 30 mm Hg the pressure drop would be 70%, if the pressure after dilation were 5 mm Hg the pressure drop would be 95% and if the pressure after dilation were 1 mm Hg the pressure drop would be 99%.

“Strut” means a wire having a generally rectangular (preferably with radiused edges) cross-section with generally flat top and bottom surfaces and having a width greater than its thickness.

“Vessel” means any vessel within a body, such as the human body, through which blood or other fluid flows and includes arteries and veins.

“Vessel flow path” means the direction of fluid flow through a vessel.

“Wire” means any type of wire, strand, strut or structure, regardless of cross-sectional dimension (e.g., the cross-section could be circular, oval, or rectangular) or shape, and regardless of material, that may be used to construct a device as described or claimed herein. Some wires may be suitable for one or more of the embodiments but not suitable for others.

### SUMMARY OF THE INVENTION

The present invention provides a device for dilating either a vessel or a structure positioned within the vessel. The device may be used in any medical application in which dilation of a vessel and/or dilation of a structure positioned within a vessel (e.g., a stent or stent graft, such as a thoracic or abdominal aortic stent graft) is desired. The device is designed so that when it is expanded it does not occlude or substantially hinder the flow of fluid through the vessel or through side vessels are connected to the vessel. The device includes a plurality of wires and has a first position in which the device is not dilated and can be moved into or retrieved from a vessel, and a second position in which the device is dilated and dilates the vessel and/or a structure within the vessel. When dilated, fluid passes through openings between the wires rather than being occluded or substantially hindered.

According to one embodiment of the invention, the device comprises a wire mesh that may be spiraled, formed in a criss-cross pattern (most preferred) or formed in any suitable pattern. The expansion and contraction of the device may be accomplished using a twisting motion (especially for a device having a spiraled wire mesh pattern) or by applying linear pressure to the device such as through a pushing or pulling motion by an operator, which compresses the device along the axis of a catheter to which it is attached and causes the device to dilate. The device can be contracted and collapsed by reversing the twisting motion or by releasing the linear pressure.

According to another embodiment of the invention, the device comprises a plurality of wires that are substantially parallel to the vessel flow path when inserted in a vessel. The expansion and contraction of such a device is preferably accomplished by applying linear pressure to the device such as through a pushing or pulling motion by an operator to compress the device and expand it, and by releasing the linear pressure to contract and collapse the device.

Any device according to the invention may be preshaped so that it automatically expands into a set position when released from a catheter sheath. It can then be dilated further or contracted by an operator in one of the manners previously described or in suitable manner. An additional advantage of this particular design is that it takes less time and operator effort to dilate or contract the device to the proper dimension for use in a procedure since the device pre-expands to a diameter close to the desired diameter.

Any device according to the invention is preferably mounted on a catheter and, utilizing the catheter, the device is positioned at the proper place within a vessel and then dilated. The catheter may be biaxial (without a cover sheath) or triaxial (with a cover sheath), which is most preferred.

The descriptions of the invention herein are exemplary only and are not restrictive of the invention as claimed.

### BRIEF DESCRIPTION OF THE DRAWINGS

FIGS. 1A-E show examples of dilation devices according to various aspects of the invention.

FIGS. 2A-C show a spiraled dilation device according to one embodiment of the invention.

FIGS. 3A-D show additional views of a spiraled dilation device according to one embodiment of the invention.

FIGS. 4A-C show a non-spiraled, dilation device according to one embodiment of the invention.

FIGS. 5A-B show another non-spiraled, dilation device according to one embodiment of the invention.

FIGS. 6A-B show a delivery and deployment system for a non-spiraled, dilation device according to one embodiment of the invention.

FIG. 7 shows a control mechanism for a dilation device according to one embodiment of the invention.

FIG. 8 is a side view of an alternate device according to the invention in a dilated position and showing a band 808 in its body.

FIG. 9 shows a side view of an alternate device according to the invention in a dilated position and having wires that are parallel to the vessel flow path when the device is positioned in a vessel.

FIG. 10 shows a side view of an alternate device according to the invention in a dilated position and having wires that are parallel to the vessel flow path when the device is positioned in a vessel.

FIG. 11 shows a side view of an alternate device according to the invention in a dilated position and having wires that are parallel to the vessel flow path when the device is positioned in a vessel.

FIG. 12 is another side view of the device of FIG. 11.

FIG. 13 is a side view of an alternate embodiment of a device according to the invention and mounted on a catheter, wherein the device comprises wires formed in a criss-cross pattern.

FIG. 14 is a close up, partial side view of the device shown in FIG. 13.

FIG. 15 is another view of the device and catheter shown in FIG. 13 illustrating how the device can be used to dilate a stent graft.

FIG. 16 is a partial, side view of an alternate device according to the invention simulating how the device conforms to a diameter disparity ratio within a vessel.

FIG. 17 is a view of the device and catheter of FIG. 13 simulating how the device conforms to a multi-vessel diameter disparity ratio.

FIG. 18 is a view of the device of FIG. 16 simulating how the device conforms to a multi-vessel diameter disparity ratio and simultaneously conforms to an asymmetrical vessel shape.

FIG. 19 is another view of the device of FIG. 16 simulating how the device conforms to a diameter disparity ratio within a vessel and showing the device covering side vessels.

FIG. 20 is another view of the device of FIG. 13 simulating the device placed in the aorta and covering the renal arteries.

FIG. 21 is another view of the device of FIG. 13 showing that it has a kink radius of at least 13.5 mm when collapsed.

FIG. 22 is another view of the device of FIG. 13 showing that it has a kink radius of at least 16 mm when fully dilated.

FIG. 23 is another view of the device of FIG. 13 showing that it has a kink radius of at least 20 mm when fully dilated.

FIG. 24 is a side, perspective view of the catheter and device of FIG. 13.

FIG. 25 is a top view of the proximal end of the catheter of FIG. 13 with the device enclosed within the catheter's outer sheath.

FIG. 26 is a top view of the proximal end of the catheter of FIG. 13.

FIG. 27 is a cross-sectional depiction of a triaxial catheter that can be used to move a device into a vessel.

### DETAILED DESCRIPTION OF PREFERRED EMBODIMENTS

A device according to the invention is for dilating a vessel and/or a structure (such as an endograft, stent or stent graft) positioned in the vessel, or alternatively may be used to simultaneously dilate two vessels or dilate a structure positioned in two vessels. The device comprises a plurality of wires and has a first position wherein it is collapsed. In this first position the device has a sufficiently small enough diameter to be positioned in a vessel where it is to be used. The device also has a second position wherein it is dilated in order to dilate a vessel and/or a structure within the vessel. When dilated the wires of the device are spaced apart to allow for the passage of fluid through the device. Thus, the device is designed so that it does not occlude or substantially hinder the flow of fluid through the vessel.

A device according to the invention may have a collapsed diameter sufficient to fit into any suitable catheter. A device according to the invention may fit into a 12 french diameter catheter, a 15 french diameter catheter, or any other catheter suitably sized for a procedure utilizing the device. The fully expanded diameter of a device according to the invention is preferably between 30 mm and 55 mm. In one embodiment the collapsed diameter is slightly less than 12 french and the fully expanded diameter is between 30 mm and 35 mm. In another embodiment the collapsed diameter is slightly less than 15 french and the fully expanded diameter is between 50 mm and 55 mm.

A device according to the invention may also be configured to have a fully expanded diameter of 15% greater than the diameter of a vessel at the location in the vessel at which the device is to be dilated.

A device according to the invention may also have any suitable length, such as any length of between 4 cm (centimeters) and 15 cm between the distal end and the proximal end when the device is in its fully collapsed position. Some preferred lengths are between 4 cm and cm, between 6 cm and 15 cm, between 8 cm and 15 cm, between 10 cm and 15 cm, and between 12 cm and 15 cm.

A device according to the invention exerts a radial force when being dilated, wherein the radial force is sufficient to dilate a stent or stent graft with which the device is used. The radial pressure can be between 5 pounds per square inch (psi) and 20 psi, between 6 psi and psi, between 7 psi and 20 psi, between 8 psi and 20 psi, between 9 psi and 20 psi, between 10 psi and 20 psi or between 15 psi and 20 psi. The radial pressure may vary within a given range depending upon the diameter of the device (e.g., the radial pressure may decrease as the diameter of the device increases). The radial pressure within a given, suitable psi range is preferably exerted over the entire working range of the device. The “working range” means all diameters of the device at which the device is expanding a stent or stent graft. In one embodiment, the measured radial force exerted at given diameters was 9.4 psi at a diameter of 20 mm, 6.7 psi at a diameter of 30 mm and 6.3 psi at a diameter of 40 mm. A device according to the invention preferably exerts a radial pressure of between 5 psi and 20 psi over at least part, and preferably over all, of its working range.

Some devices according to the invention are also sufficiently compliant (or flexible) so that when placed in a vessel and dilated they conform to the dimensions of the vessel even when the vessel dimensions are not uniform. In particular, some devices of the present invention can conform to one or more diameter disparity ratios of between 1.2:1 and 3.4:1 and some devices according to the invention can conform to one or more multi-vessel diameter disparity ratios of between 1.2:1 and 3.4:1.

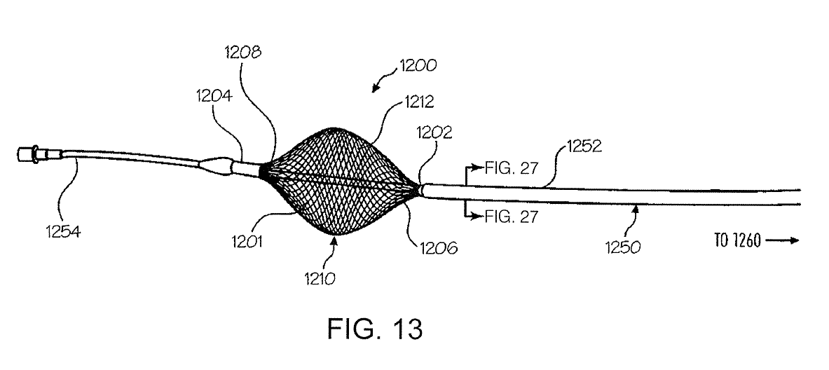

The wires used in a device according to the invention may be of any suitable size, shape, thickness and material. For example, all or some of the wires may have a generally circular cross-section and have a diameter of between 0.008″ and 0.018″. Alternatively, all or some of the wires may include one or more struts that have a thickness of between 0.008″ and 0.018″ and a width of between 0.020″ and 0.050″. A wire may be comprised of stainless steel, nitinol, cobalt, chromium or any suitable metal, plastic or other suitable material. In one preferred embodiment, the wire is comprised of nitinol, has a generally circular cross section and a diameter of about 0.015″. In this embodiment, the wires are formed in a criss-cross pattern (as shown in FIGS. 13-20).

The device may have any suitable density of wires and the wires may be formed in any suitable pattern, such as in a criss-cross pattern or in a non-overlapping pattern in which the wires are parallel to vessel flow path (as shown in FIGS. 9-12).

If a device according to the invention has wires that are parallel (as used in this context, “parallel” means the wires are substantially parallel to one another) to the vessel flow path, the device may have between four and twenty-four wires, or may have more than twenty-four wires. In various embodiments, a device according to the invention includes, respectively, four wires, five wires, six wires, seven wires, eight wires, nine wires, ten wires, eleven wires, twelve wires, thirteen wires, fourteen wires, fifteen wires, sixteen wires, seventeen wires, eighteen wires, nineteen wires, twenty wires, twenty-one wires, twenty-two wires, twenty-three wires and twenty-four wires. The maximum distance between each wire in such a device can vary depending upon the number of wires, the width of the wires and the proposed use of the device, but generally the maximum distance between wires will be between 1 mm and 100 mm when the device is fully dilated. In various embodiments of the device, the maximum distance is, respectively, no greater than 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm or 100 mm.

If a device according to the invention includes wires in a criss-cross pattern, each of the largest spaces between the wires when the device is fully dilated could have an area of between 1 mm² and 400 mm², including areas of 1 mm², 2 mm², 4 mm², 10 mm², 25 mm², 50 mm², 75 mm², 100 mm², 150 mm², 200 mm², 250 mm², 300 mm², 350 mm², and/or 400 mm² or areas within that range. It is also possible that the area of the largest spaces could be larger than 400 mm² or smaller than 1 mm², as long as the device falls within the scope of one of the claims and works for its intended purpose of dilating a vessel or dilating a structure within a vessel without occluding or substantially hindering fluid flow through the vessel.

A device according to the invention may also have spaces between the wires that are greater in the central portion of the device than at the ends of the device, as illustrated, for example, in FIGS. 9-15 and 17-20.

A device according to the invention may be constructed to any suitable size or in any suitable manner to accommodate a particular vessel, including veins and arteries (e.g., the abdominal aorta, aortic arch, the ascending aorta, the descending aorta, an iliac artery, or a renal artery). For example, the device may be used in wall apposition of a thoracic and/or abdominal endoluminal grafts, which means it expands to position at least a portion of a stent graft snugly (without a sheath) against the artery wall.

A device may be introduced into a vessel using either a biaxial (without a sheath) or triaxial (with a sheath) catheter, which is typically inserted over a guide wire. Optionally, the device includes one or more radio opaque markers that assist an operator in locating the device once in a vessel, although a device according to the invention can generally be seen using fluoroscopy without the need for radio opaque markers.

When dilated, a device according to the invention does not occlude or substantially hinder the flow of fluid through a vessel or into a side vessel because the fluids flow through the spaces (or openings) between the wires. In a pressure monitoring test using water as the fluid and a plastic tube to simulate the aorta the pressure drop within a vessel and downstream of a dilated device as generally shown in FIGS. 13-20 was measured as less than 1%. This test measured the flow lengthwise through the device, wherein the water had to flow through both the proximal end and distal end of the device. Thus, the water had to flow through the smallest openings in the device, which in the embodiment tested were located at the distal end and the proximal end. It is therefore believed that flow into a side vessel, wherein fluid would flow through the smaller openings in the distal end of the device and then through larger openings in the body portion of the device and into the side vessel, would be less hindered than flow lengthwise through the device. Accordingly, the pressure drop due to the dilation of a device according to the invention, either measured downstream of the proximal end of the device or measured in a side vessel covered by the device (such as when the device is in the aorta and covers one or both renal arteries), would be less than 70%, less than 60%, less than 50%, less than 40%, less than 30%, less than 20%, less than 10%, less than 5%, less than 2%, and/or less than 1%.

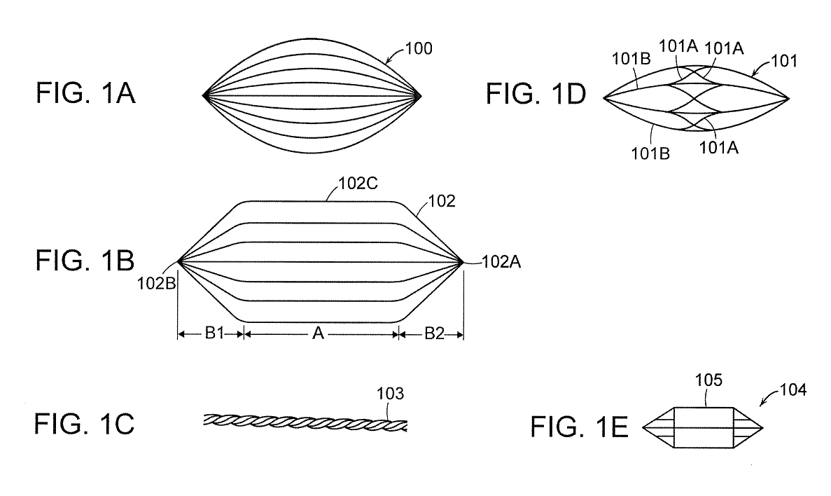

Reference will now be made to preferred embodiments of the invention, examples of which are illustrated in the accompanying drawings, wherein the purpose is to describe certain examples of the invention and not to limit the scope of the claims. FIGS. 1A-E show examples of spiraled devices according to various aspects of the invention. These devices are preferably dilated and collapsed by winding (to contract) and unwinding (to dilate) a plurality of wires that are preferably formed in a spiraled pattern. Device 100 shown in FIG. 1A is a generally oval-shaped dilation device in a dilated position. Device 102 shown in FIG. 1B is a dilation device with a substantially-linear section A of wires in the middle of device 102, while the wires in end sections B1 and B2 are at an angle so that they converge at approximately the same point at each respective end 102A, 102B on either side of device 102. In this way, section A of dilation device 102 may exert more even pressure against a vessel and/or structure within a vessel. In this example, the substantially-straight section A is approximately 3 cm in length, while each of the end sections B1 and B2 is approximately 1 cm in length. However, the device may be of any suitable size or shape and be constructed in any manner.

Device 101 shown in FIG. 1D, is spiraled, shown in a dilated position and includes support members 101A between wires 101B. Support members 101A provide additional strength to device 101.

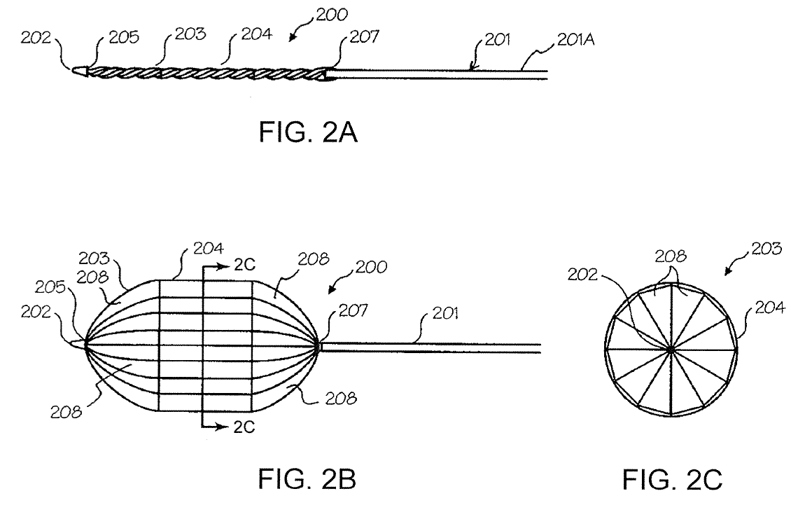

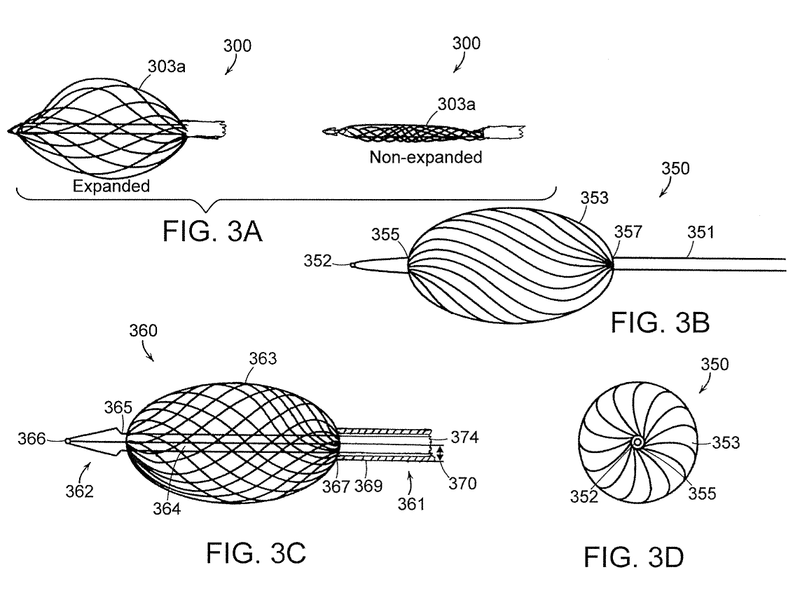

Device 103 shown in FIG. 1C is an exaggerated view of wires in a spiraled dilation device such as device 100 when the wires are in a spiraled position. In this position, the diameter of the dilation device is reduced, allowing for insertion into a blood vessel. Unspiraling the wires causes the device to dilate, as shown in FIGS. 1A-1C. Other embodiments of a spiraled dilation device will be discussed further with regard to FIGS. 2A-C and FIGS. 3A-D.

Any device according to the invention may utilize a lining, such as lining 105 shown in FIG. 1E. A lining such as lining 105 may be positioned on part of the exterior surface and/or interior surface of a device, such as 104. The use of a lining (a) provides a more even surface (depending upon the nature of the device with which it is used) for exerting pressure during the dilation process, and/or (b) helps to prevent the wires of the device from becoming entangled with exposed wires on a stent or stent graft.

Lining 105 is preferably made from a permeable material, which would be important if the lining is positioned such that it could occlude or seriously hinder blood flow. However, impermeable materials may used if the lining is not positioned where it could seriously hinder blood flow. For example, in device 104, even if an impermeable material is used for the lining, blood will still flow through the gaps between the wires at each end of the device. So as long as device 104 is not positioned so that it blocks a side vessel, or an impermeable membrane on device 104 is not positioned so that it blocks a side vessel, an impermeable material could be used. Any suitable material may be used for liner 105 and examples of preferable lining materials include, but are not limited to, polyurethane, PTFE (polytetrafluoroethylene), nylon, or any material used in carotid embolic protection devices.

FIGS. 2A-C show an assembly 200 according to an embodiment of the invention. FIG. 2A shows spiraled dilation device 203 in a first position for insertion into a blood vessel. Assembly 200 also includes a biaxial catheter 201 (biaxial catheter does not include a catheter sheath) with a distal tip 202. Catheter 201 (and any catheter used with a device as described or claimed herein) may be made of any material suitable for insertion into a vessel and may be sized for a particular vessel. Catheter 201 has an outer tube 201A, a central tube (not shown) running the length of the catheter. The central tube includes a wire port (not shown) in distal tip 202 and a lumen (not shown) for receiving a guide wire. Catheter 201 is inserted into a vessel by being guided over a guide wire going through the wire port and through the lumen in the central tube. Catheter 201 may be triaxial, in which case there would be a catheter sheath over device 203 (as described below).

Dilation device 203 is affixed to catheter 201 at point 205 and also at point 207. As shown in FIG. 2A, dilation device 203 is spiraled around the central tube in catheter 201 in a first position. In this position, catheter 201 and dilation device 203 are positioned to be inserted into the vessel. Dilation device 203 may optionally include a lining 204, which may be one of the same types of linings as discussed above.

FIG. 2B shows device 203 in an expanded position. Dilation device 203 is expanded by exerting a twisting motion on either outer tube 201A (preferred) or on the central tube, while keeping the other of the two tubes relatively still so that device 203 can expand. Because dilation device 203 is affixed at point 205 to the central tube and at point 207 to outer tube 201A, a twisting motion applied to outer tube 201A, while keeping the central tube relatively still (at least with as little motion as necessary to allow device 203 to expand), will unspiral and dilate device 203. Optionally, the twisting motion will be applied to the central lumen.

FIG. 2C shows a cross-sectional view taken along line A-A when dilation device 203 is in an expanded position. As can be seen, lining 204 provides for a more substantially uniform surface than would the wire mesh of dilation device 203 alone. Gaps 208 between the wires of dilation device 203 allow fluid to flow through device 203.

FIGS. 3A-3D show additional views of an assembly including a spiraled dilation device according to one embodiment of the invention. FIG. 3A shows a spiral mesh structure rather than the straighter, cage-like structure of FIGS. 2A-C. The spiral mesh shown in FIGS. 3A-3D has a greater wire density when expanded than the structure shown in FIGS. 2A-C. The wire density (i.e., the number of wires in a given area) used in a dilation device may be varied for different applications. In general, the denser the wire mesh when a device is dilated, the more surface area available to press against a blood vessel and/or structure within the blood vessel.

FIG. 3A shows dilation device 303a in an expanded position and in a non-expanded position. FIG. 3B shows an assembly 350 including an expanded spiral mesh device 353 and catheter 351. Device 353 is affixed to catheter 351 at affixation points 355 and 357, catheter 351 also has a distal tip 352.

FIG. 3C shows a partial, sectional side view of an assembly 360 with a dilation device 363 having a wire mesh structure, and a catheter 361 with a distal tip 362. While not necessary, a tapered front end on distal tip 362 allows for easier insertion into a vessel. At the end of distal tip 362 is a wire port 306, which leads to a lumen, for insertion of catheter 361 over a guide wire 370. The proximal end of distal tip 362 may have a reverse taper towards affixation point 365. Affixation point 365 is the point at which the distal end of dilation device 363 connects to central tube 364. Affixation point 367 is the point at which the proximal end of dilation device 303 connects to outer tube 369, which is positioned coaxially around central tube 364. Dilation device 363 is expanded by twisting outer tube 369 (or alternatively by twisting central tube 364).

FIG. 3D shows a front view of device 350, which was previously described.

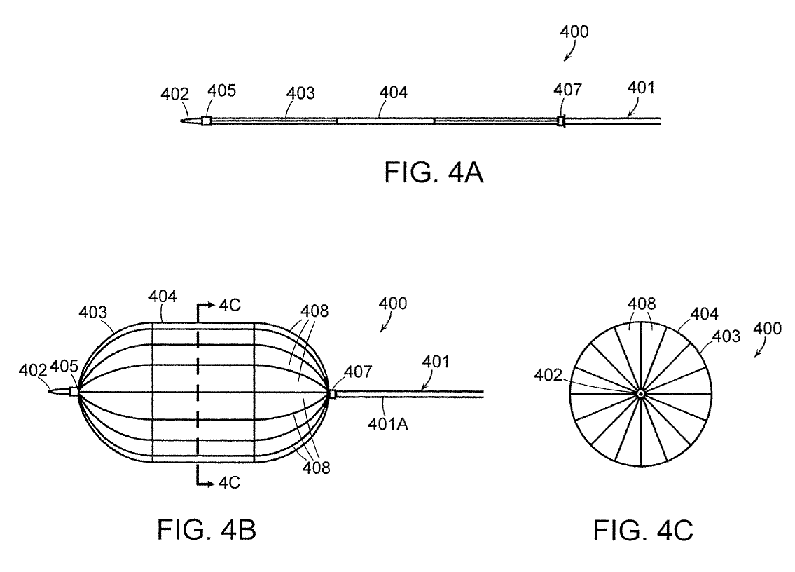

FIGS. 4A-C show an assembly 400 having a non-spiraled, expansive dilation device according to one embodiment of the invention. FIG. 4A shows a non-spiraled, dilation device 403 in a first position for insertion into a blood vessel. Assembly 400 also includes a catheter 401 with a distal tip 402. Catheter 401 may be any device having a central lumen and being capable of insertion into a blood vessel over a guide wire. Catheter 401 has a central tube (not shown) with a wire port (not shown) in distal tip 402 that communicates with a lumen in the central tube. Catheter 401 is inserted into a blood vessel over a guide wire going through the wire port and into the lumen.

Dilation device 403 is affixed to catheter 401 at point 405 and also at point 407. As shown in FIG. 4A, dilation device 403 is not spiraled. That is, each wire of dilation device 403 is substantially parallel to the other wires and runs in a substantially straight line from affixation point 405 (on a central tube, which is not shown) to affixation point 407 on outer tube 401A. In this first position shown in FIG. 4A, the catheter 401 and dilation device 403 are insertable into a vessel. Dilation device 403 may optionally include a lining 404 as discussed above with reference to FIG. 1, which in this embodiment is on the inside surface of dilation device 403.

FIG. 4B shows device 400 in an expanded position. Dilation device 403 is expanded by exerting linear pressure via catheter 401 (e.g., a push-pull motion). Because dilation device 403 is affixed at points 405 and 407, a linear motion applied to one tube of catheter 401 (such as by pulling the central tube or pushing outer tube 401A) will expand device 403. As can be seen in FIG. 4B, the use of optional lining 404 creates a substantially uniform surface for dilating blood vessels and structures.

FIG. 4C shows a sectional view taken along line A-A when dilation device 403 is in the expanded position. Gaps 408 between the wires of dilation device 403 allow fluid to flow through device 403.

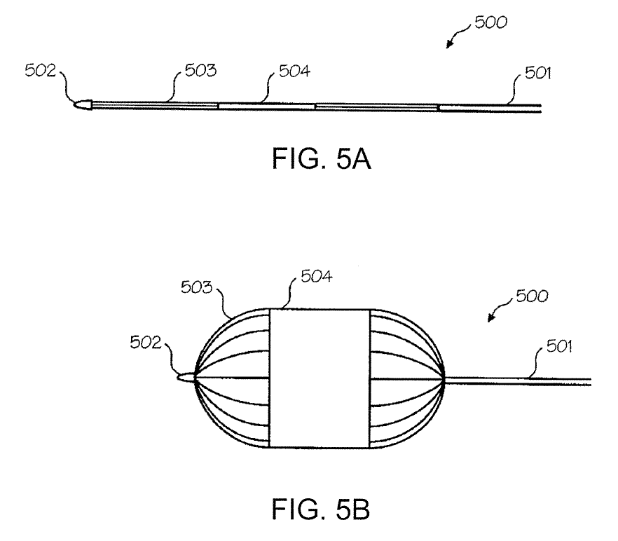

FIG. 5 shows assembly 500 including a non-spiraled, expansive dilation device 503 according to one embodiment of the invention. Assembly 500 also includes a catheter 501 having a distal tip 502. Assembly 500 has the same structure as assembly 400 except that liner 504 is placed on the outside of dilation device 503.

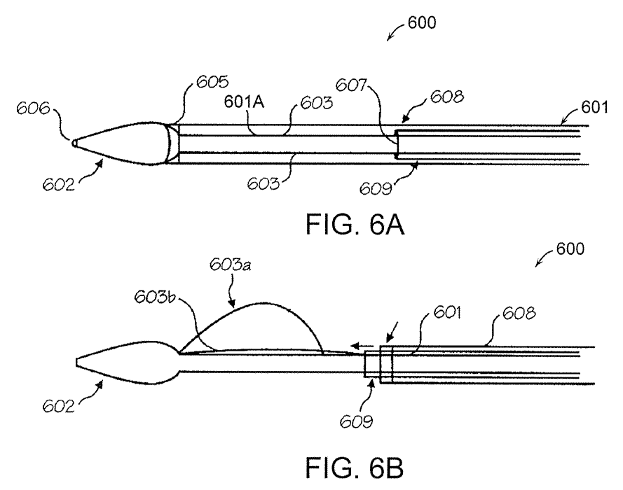

FIGS. 6A-B show an assembly 600 for a non-spiraled, dilation device according to an embodiment of the invention. Triaxial catheter 601 includes a central tube 601A, an outer tube 609 and a catheter sheath 608. Wire port 606 may be constructed to fit over any size guide wire (e.g., port 606 may be a 0.038″ diameter wire port). Affixation point 605 is where the distal end of dilation device 603 attaches to central tube 601A. Outer tube 609 is positioned coaxially around central tube 601A and the proximal end of dilation device 603 attaches to outer tube 609 at affixation point 607. Catheter sheath 608 is positioned coaxially around outer tube 609 and can be moved towards tip 602 to cover device 603 or away from tip 602 to expose tip 603. Catheter sheath 608 may include radiopaque markers to indicate when device 603 has cleared the treatment zone.

FIG. 6B shows dilation device 603 in two positions. In position 603a, dilation device 603 is expanded. The expansion is accomplished by pushing or otherwise moving (such as by using a screw mechanism) outer tube 609 forward (preferred) while keeping central tube 601A relatively stationary or central tube 601A backward while keeping outer tube 609 relatively stationary. In this manner the proximal end of dilation device 603 and the proximal end of dilation device 603 are moved towards each other and the wires of dilation device 603 expand outward. In position 603b, the wires of dilation device 603 remain at essentially their smallest diameter and close to central tube 601A. If device 603 had been expanded, it is moved to the position shown in position 603b by increasing the distance between the distal end and proximal end of device 603 by either pulling outer tube 609 back or pushing central tube 601A forward.

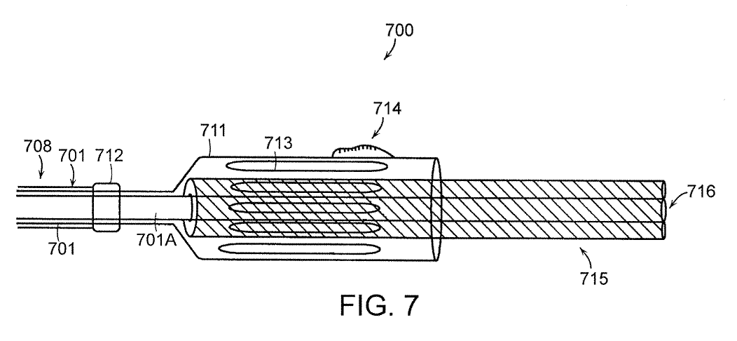

FIG. 7 shows a control mechanism 700 for a dilation device according to one embodiment of the invention. Control mechanism 700 is the hand-held portion of a dilation assembly (which in this embodiment is a catheter that includes the controls and the device) and may be used with both spiraled and non-spiraled dilation devices. In the case of a non-spiraled, dilation device, handle 711 is attached to catheter sheath 708 through hemostatic valve 712. For both spiraled and non-spiraled dilation devices, central tube 701A of catheter 701 runs through handle 711 and has a wire port 716 at its distal end that communicates with a lumen.

As shown in FIG. 7, handle 711 is a nut-type handle that is either fused to an outer sheath and may be twisted (for a spiraled dilation device) or pushed/pulled (for a non-spiraled, expansive dilation device) to engage or disengage a dilation device. Handle 711 may include surface texturing 713 for easier grip. Handle 711 may also include a threaded, bolt-type fixation handle 715 that is fused to catheter 701. This allows for execution of a twisting motion for spiraled dilation devices. Handle 711 may also include a thumb-controlled quick release 714. Quick release 714 disengages handle 711 from the bolt-type fixation handle, allowing push/pull motions to be exerted on the handle and any attached sheaths and/or catheters (e.g., for engaging non-spiraled dilation devices).

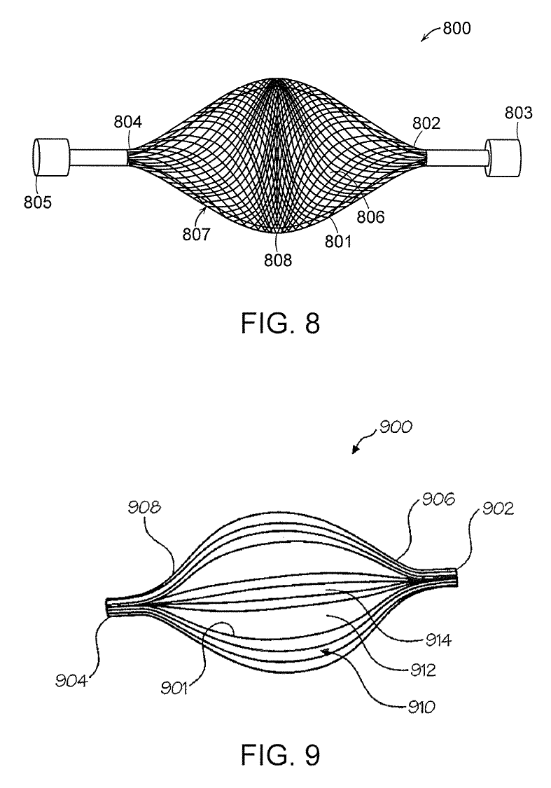

FIG. 8 shows an alternate device 800 according to the invention that is shown in a dilated position. Device 800 is comprised of wires 801 and includes a proximal end 802 retained by a retention member 803 and a distal end 804 retained by a retention member 805. As used herein, the distal end and the proximal end are the parts of the device that extend about 15 mm from each respective retention member. Device 800 has a body portion 807 positioned between ends 802 and 804 and spaces 806 are formed between wires 801 when device 800 is dilated as shown. Spaces 806 are preferably (but not necessarily) greater between wires 801 in body portion 807 than the spaces 806 between the wires 801 at end 802 or end 803 when device 800 is dilated. A band of wires 850 may be formed near the center of body portion 807 to add greater radial strength, and the spaces between the wires 801 in such a band are typically smaller than the spaces between the wires 801 in other parts of body portion 807.

FIG. 9 shows a device 900 according to the invention that is in the dilated position and comprises a plurality of wires 901. In this embodiment each wire 901 is parallel to the other wires 901 (in this context “parallel” means substantially parallel). Each of the wires 901 is also parallel to the vessel flow path when device 900 is inserted into a vessel (again, in this context, “parallel” means substantially parallel). Device 900 as shown is formed by slitting a tube and has unslitted ends 902, 904 a proximal end 906 and a distal end 908. Device 900 has a body portion 910 between proximal end 906 and distal end 908. As shown, wires 901 are formed in three-wire groups with distances 912 between the groups and distances 914 between wires in each group. Distances 912 are greater than distances 914 and each of the respective distances 912 and 914 are greater in body portion 910 than they are at either proximal end 906 or distal end 908.

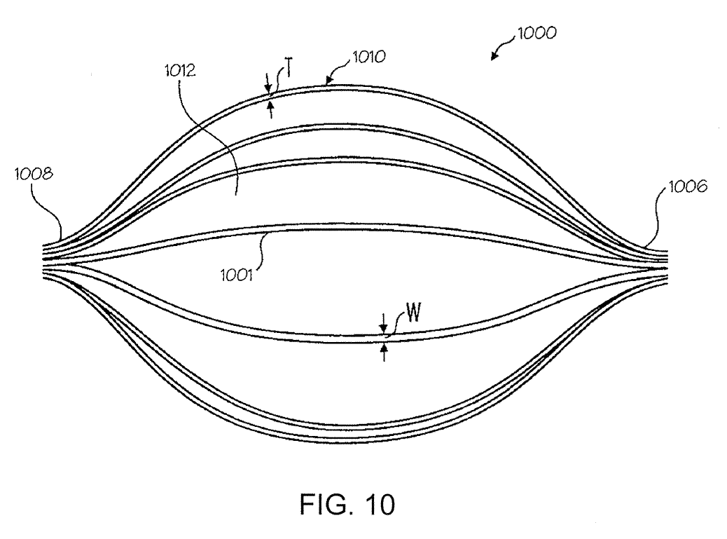

FIG. 10 shows a device 1000 that is in a dilated position. Device 1000 comprises a plurality of wires 1001 and is preferably formed by slitting a tube and leaving the ends of the tube (not shown in this Figure) unslit. In this embodiment each of the wires 1001 is parallel (in this context “parallel” means substantially parallel) to the other wires 1001 and each of the wires 1001 is also parallel (again, in this context, “parallel” means substantially parallel) to the vessel flow path when device 1000 is positioned in a vessel. Each wire 1001 is preferably a strut having a generally rectangular cross section and preferably having a width greater than its thickness. The width could be any suitable width but is preferably between 0.020″ and 0.050″ and the thickness could be any suitable thickness but is preferably between 0.008″ and 0.018″. Device 1000 has a proximal end 1006, a distal end and 1008 and a body portion 1010. There is a distance 1012 between wires 1001 and in this embodiment the distance 1012 is greater in body portion 1010 than in either proximal end 1006 or distal end 1008.

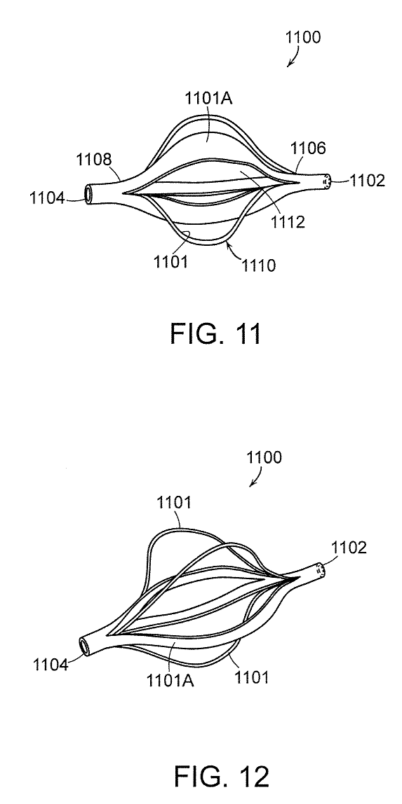

FIGS. 11 and 12 show a device 1100 according to the invention that is in a dilated position and that comprises a plurality of wires 1101. In this embodiment each wire 1101 is parallel to the other wires 1101 (in this context “parallel” means substantially parallel). Each of the wires 1101 are also parallel to the vessel flow path when device 1100 is inserted into a vessel (again, in this context, “parallel” means substantially parallel). Device 1100 as shown is formed by slitting a tube and has unslitted ends 1102 and 1104 (shown in FIG. 12) that are connected, respectively, to proximal end 1106 and distal end 1108. Device 1100 has a body portion 1110 between proximal end 1106 and distal end 1108. Device 1100 has two types of wires, wires 1101 and 1101A. As shown wires 1101 are slender, having a preferred width of between about 0.008″ and 0.014″ whereas wires 1101A are wider and have a width of between about 0.020″ and 0.025.″ Wires 1101 also extend further from the center of body portion 1110 than do wires 1101A. In this embodiment wires 1101 and 1101A function together to apply even pressure to a substantial area of a vessel and/or apply even pressure to a substantial area of a structure to be positioned within a vessel.

FIG. 13 shows a device 1200 according to the invention that is mounted on a catheter 1250. Catheter 1250 is of a triaxial design generally known in the art and includes a catheter sheath 1252, a proximal end 1260 (best seen in FIG. 26), which is outside of the patient's body during a procedure and is juxtaposed the operator when catheter 1250 is in use, and a distal end 1254 that is inserted into the body.

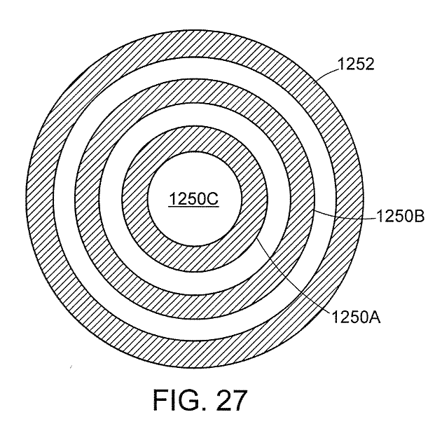

FIG. 27 shows a cross-sectional view of catheter 1250 taken through line C-C of FIG. 13. Catheter 1250 includes, but is not limited to, three preferably coaxial tubes; central tube 1250A, outer tube 1250B and catheter sheath 1252. In this embodiment, central tube 1250A extends essentially the entire length of catheter 1250 and has a central lumen 1250C for receiving a guide wire (not shown). Central tube 1250A extends through device 1200 and is attached to device 1200 at end 1204. Outer tube 1250B is positioned over central tube 1250A and extends to end 1202 of device 1200 where it is connected to end 1202. Catheter sheath 1252 has a length sufficient to cover device 1200.

In operation the assembly including device 1200 and catheter 1250 is placed into a vessel with catheter sheath 1252 at least partially covering device 1200 to help retain it in its collapsed position and to allow for ease in directing the catheter and device through the vessel.

Once device 1200 is properly positioned in a vessel, catheter sheath 1252 is pulled back to expose device 1200. Device 1200 can then be dilated by either pushing outer tube 1250B, pulling central tube 1250A or by simultaneously pushing outer tube 1250B and pulling central tube 1250A. As previously explained, the tube that is not being pushed or pulled must remain stable enough so that the distance between retention ends 1202 and 1204 decreases and device 1200 expands.

If a device according to the invention were being used to position a structure in the vessel, the structure (such as a stent or stent graft) could be mounted on the device in a typical manner known to those in the art so that as the device dilates the structure is dilated.

Utilizing catheter 1250 (or any suitable biaxial or triaxial catheter) a device, such as device 1200 or 1300, is dilated by moving the distal and proximal ends of the device towards each other. The device is contracted and collapsed by releasing the force pushing the two ends together and/or by moving the two ends apart.

Alternatively, any device according to the invention may be preformed in a dilated position and compressed into a collapsed position when covered by catheter sheath 1252. When catheter sheath 1252 is removed the preformed device would immediately expand to its dilated position and then could be contracted or further dilated by an operator utilizing the catheter in one of the manners described.

In FIG. 13, device 1200 is shown in its dilated position and it comprises a plurality of wires 1201 that are formed in a criss-cross pattern. Device 1200 has retention ends 1202 and 1204 that may be formed as part of catheter 1250, a proximal end 1206, a distal end 1208 and a body portion 1210. Spaces 1212 are formed between wires 1201 and can be of any suitable size, e.g., between about 1 mm² and about 400 mm². As shown, spaces 1212 are larger in body portion 1210 that in either proximal end 1206 or distal end 1208.

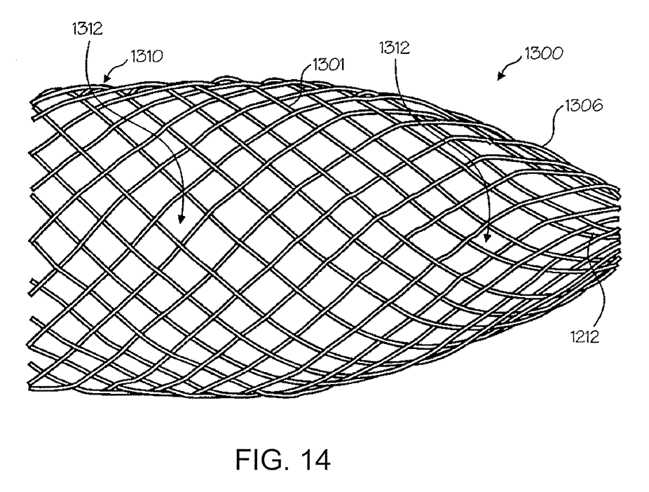

FIG. 14 is a close-up, partial side view of an alternate device 1300 showing proximal end 1306 and part of body portion 1310. As can be seen spaces 1312 between wires 1301 are smaller at proximal end 1306 than at body portion 1310.

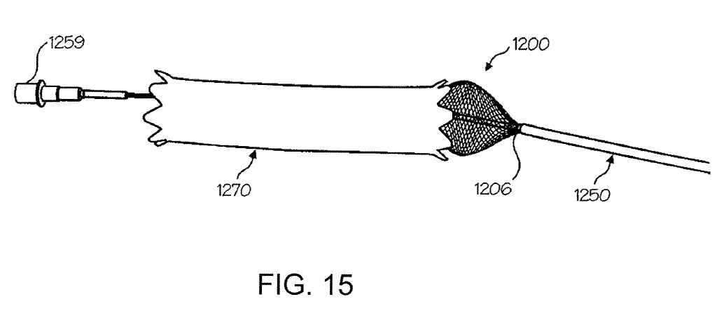

FIG. 15 generally illustrates how device 1200 can be utilized to dilate a stent graft 1270, which is shown in a dilated position. Device 1200 is positioned inside of the portion of the stent graft that will be compressed against a vessel wall to anchor the stent graft in place. As the device is expanded it presses the stent graft against the vessel wall.

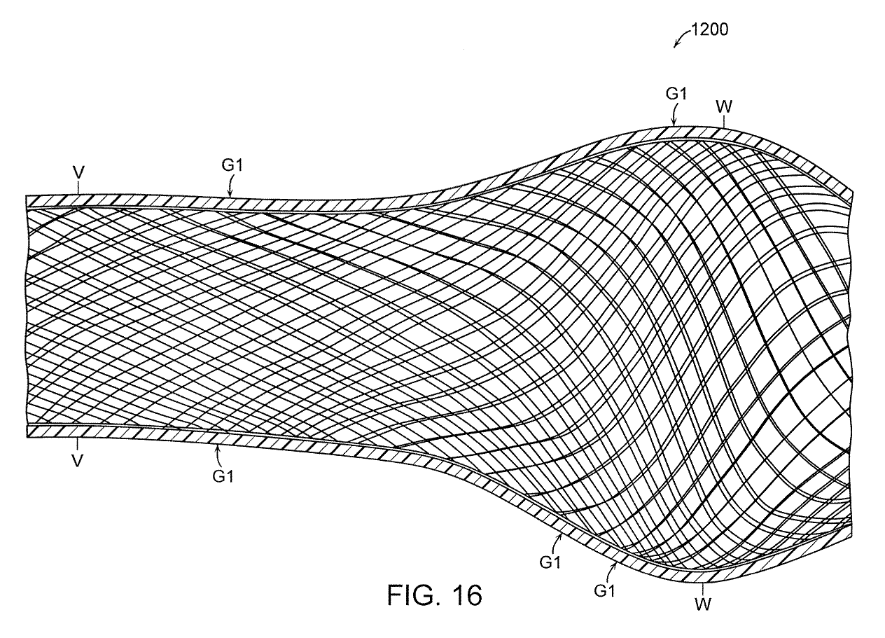

FIG. 16 shows device 1300 dilated in a plastic model G1 to simulate device 1200 conforming to a diameter disparity ratio of approximately 1.8:1 in a vessel. Device 1300 is pressed against the entire interior wall of model G1 from at least position V to a position past position W.

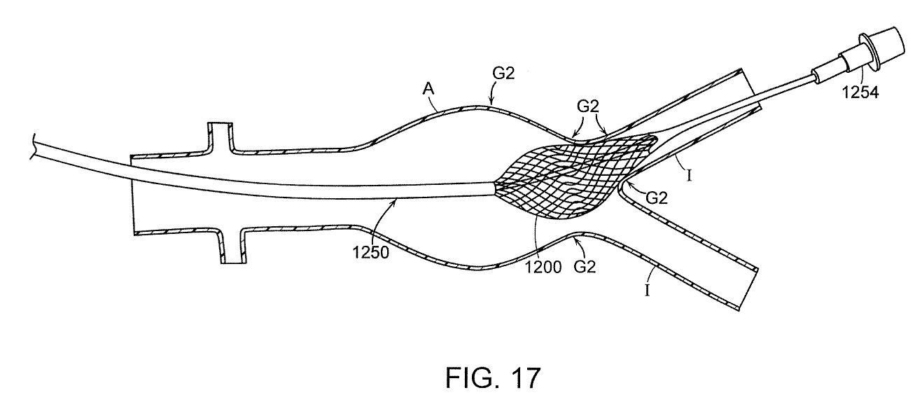

FIG. 17 shows device 1200 and catheter 1270 in a plastic model G2 that simulates the aorta A and the iliac arteries I. In this Figure device 1200 is simultaneously positioned in the aorta and an iliac artery and is conforming to a multi-vessel diameter disparity ratio of about 2.0:1.

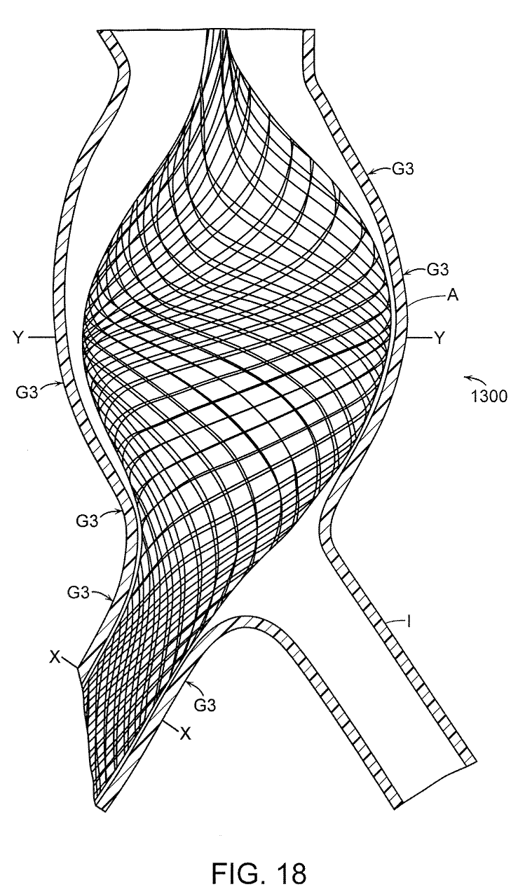

FIG. 18 shows a device 1300 in accordance with the invention that is dilated in a plastic model G3 to simulate device 1300 being dilated simultaneously in the aorta A and an iliac artery I. In this Figure device 1300 is conforming to a multi-vessel diameter disparity ratio of about 3.4:1. Device 1300 is pressed against the entire interior wall of model G3 (except for one of the simulated iliac arteries I that does not include device 1300) from at least position X to a position past position Y.

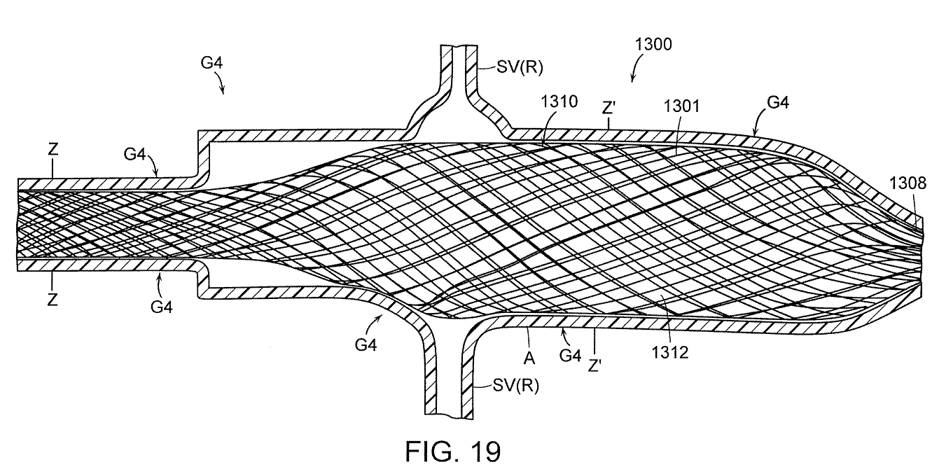

FIG. 19 shows device 1300 with wires 1301, proximal end 1308 and spaces 1312 between wires 1301. Device 1300 is dilated in a plastic model G4 to simulate device 1300 being dilated in aorta A and covering side vessels SV(R) that simulate the renal arteries. As can be seen, fluid would flow through the spaces 1312 at proximal end 1308, through the aorta and into the side vessels through spaces 1312 in body portion 1310. In this Figure, device 1300 is also conforming to a vessel diameter disparity ratio of about 2.0:1. Device 1300 is pressed against the entire interior wall of model G4 (except for the simulated renal arteries SV(R) shown as side vessels that are covered by device 1300) from at least position Z to a position past position Z′.

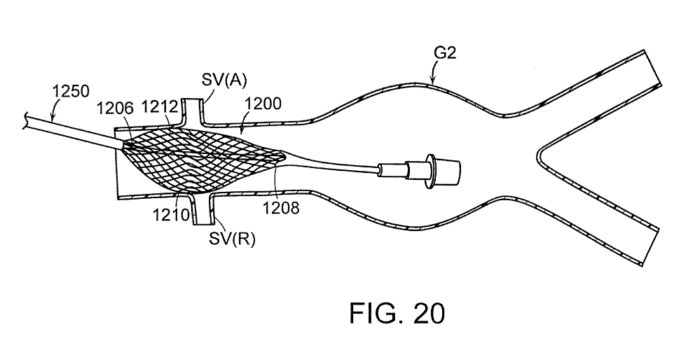

FIG. 20 shows device 1200 and catheter 1250 positioned in a plastic model G2 to simulate device 1200 being positioned and dilated in the aorta and covering side branches, such as the renal arteries SV(R). The spaces 1212 between the wires 1201 in device 1200 allow fluid to flow through the aorta and into the side vessels when device 1200 is dilated.

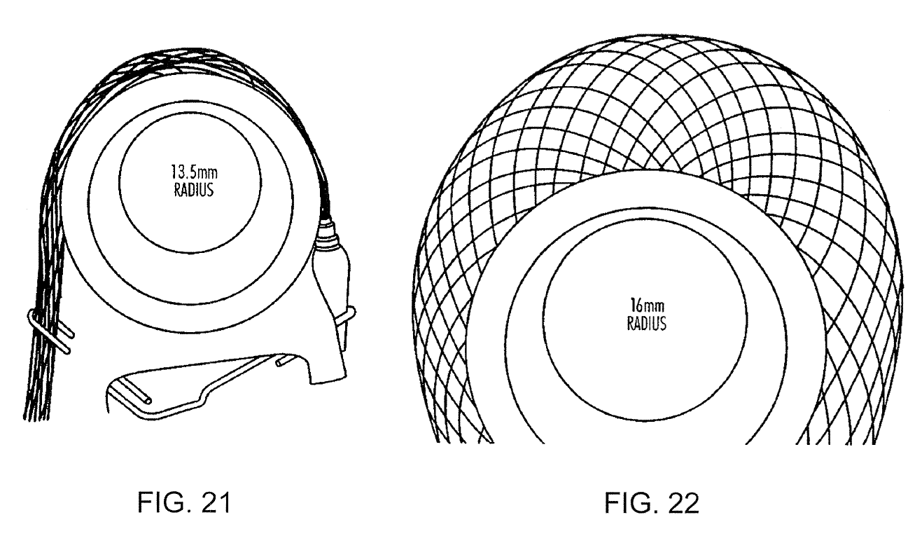

FIG. 21 shows the device of FIG. 13 in its collapsed position and having a kink radius of 13.5 mm.

FIG. 22 shows the device of FIG. 13 in its fully dilated position and having a kink radius of 16 mm.

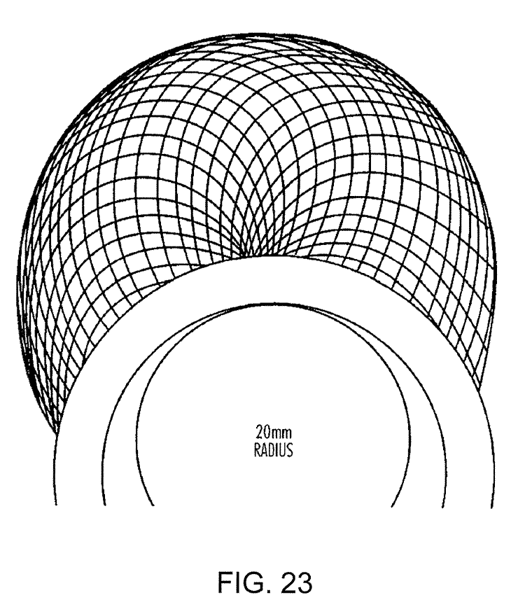

FIG. 23 shows the device of FIG. 13 in its fully dilated position and having a kink radius of 20 mm.

A device according to the present invention thus may have a kink radius of 13.5 mm or greater before being dilated. This includes one or more of a kink radii of 14.0 mm, 15.0 mm, 16.0 mm, 17.0 mm, 18.0 mm, 19.0 mm, 20.0 mm and greater. Further, a device according to the present invention may, when fully dilated, have a kink radius of 16.0 mm or greater. This includes one or more of the kink radii of 17.0 mm, 18.0 mm, 19.0 mm, 200 mm, 21.0 mm, 22.0 mm, 23.0 mm, 24.0 mm, 25.0 mm and greater.

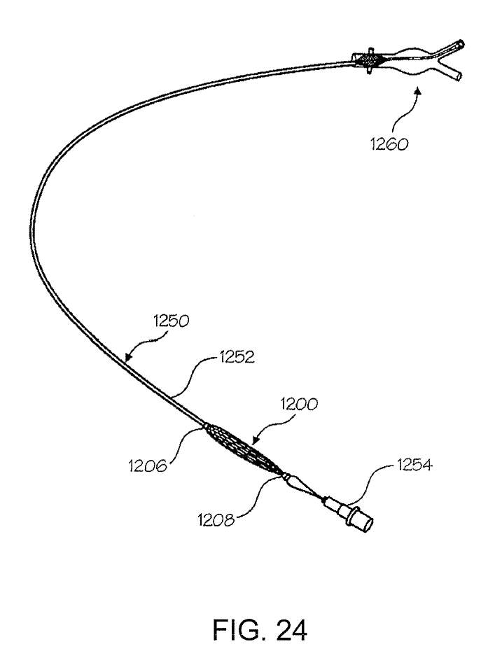

FIG. 24 shows the catheter 1250 of FIG. 13 that includes device 1200. Catheter 1250 has a proximal end 1254 that is inserted into a vessel during use, and a distal end 1260 that remains outside of the vessel and is used by an operator to position, release and dilate device 1200.

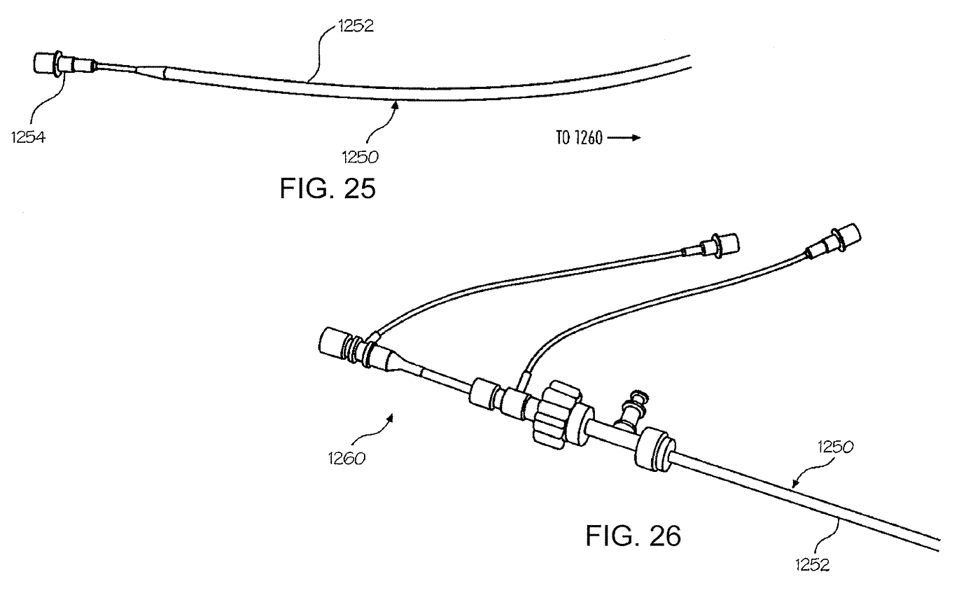

FIG. 25 shows proximal end 1254 of catheter 1250.

FIG. 26 shows distal end 1260 of catheter 1250.

Other embodiments of the invention will be apparent to those skilled in the art from consideration of the specification and embodiments disclosed herein. Thus, the specification and examples are exemplary only, with the true scope and spirit of the invention set forth in the following claims and legal equivalents thereof.

1. A device for dilating a vessel or a structure positioned within a vessel, the device comprising a plurality of wires that are spaced apart when the device is dilated to allow for passage of fluid through the device, the device being sufficiently compliant so that when placed into a vessel and dilated it can conform to at least one diameter disparity ratio of between about 1.2:1 and about 3.4:1.

2-13. (canceled)

14. The device of claim 1 wherein each of the wires are parallel to a vessel flow path when in the vessel.

15. The device of claim 1 wherein at least some of the wires form a criss-cross pattern.

16. The device of claim 1 wherein the wires are comprised of nitinol.

17. The device of claim 1 wherein the wires have a circular cross-sectional and a diameter of between about 0.008″ and about 0.018″.

18. The device of claim 1 further including a permeable membrane on at least part of the device.

19. The device of claim 18 further comprising an outer surface and the permeable membrane is positioned on the outer surface.

20. The device of claim 1 wherein the device has a fully collapsed diameter of less than 12 french and a fully expanded diameter of between about 30 mm-about 35 mm.

21. The device of claim 1 wherein the device has a fully collapsed diameter of less than about 15 french and a fully expanded diameter of between about 50 mm and about 55 mm.

22. The device of claim 1 wherein the device exerts a radial pressure of between about 5 psi and about 20 psi over at least part of its working range.

23. The device of claim 1 wherein the device exerts a radial pressure of between about 5 psi and about 20 psi over its entire working range.

24. The device of claim 1 wherein the device has a length of between about 4 cm and about 15 cm.

25. An assembly comprising:
the device of claim 1; and
a catheter comprising a catheter sheath that at least partially encloses the device during insertion of the device into the vessel, the device being preshaped to automatically expand when released from the catheter sheath.

26. The device of claim 1, wherein the device is adapted to dilate two vessels or a structure positioned within two vessels, the device being sufficiently compliant so that when it is simultaneously positioned in each of the two vessels the device can conform to at least one multi-vessel diameter disparity ratio of between about 1.2:1 and about 3.4:1.

27-67. (canceled)

68. A device for dilating a vessel or a structure within a vessel, the device comprising a plurality of wires that are spaced apart when the device is dilated to allow for passage of fluid through the device, the wires being parallel to each other and being parallel to a vessel flow path when in the vessel.

69-96. (canceled)

97. A device for dilating a vessel or a structure within a vessel, the device comprising a plurality of wires that are spaced apart when the device is dilated to allow for passage of fluid through the device, wherein the device before being dilated has a kink radius of greater than or equal to about 13.5 mm.

98. The device of claim 97 wherein at least some of the wires are comprised of nitinol.

99. The device of claim 97 wherein the wires have a circular cross section and are between about 0.008″ and about 0.018″ in diameter.

100. The device of claim 97 wherein at least some of the wires form a crisscross pattern, the device has a body portion and the space between at least some of the wires in the body portion is between about 2 mm² and about 400 mm² when the device is fully dilated.

101. The device of claim 97, wherein the device when collapsed has a kink radius of between about 13.5 mm and about 16.0 mm.

102. The device of claim 101 wherein the device has kink radius of between about 16.0 mm and about 20.0 mm when the device is fully dilated.

103-105. (canceled)

106. An assembly including a device for dilating a vessel or structure within a vessel, the device comprising a plurality of wires that are spaced apart when the device is dilated to allow for the passage of fluid through the device, wherein when the device is positioned in a vessel and against a side vessel, and the pressure drop in the side vessel is less than 70% when the device is dilated, as measured in a pressure monitoring test utilizing water.

107-120. (canceled)

121. The device of claim 26 further comprising a distal end, a proximal end and a body portion between the distal end and the proximal end and spaces between the wires wherein at least some of the spaces between the wires in the body portion are larger than the spaces between the wires at the distal end or the proximal end when the device is dilated.

122-126. (canceled)

127. An assembly including a device for dilating a structure within a vessel, the device comprising a wire mesh body and including:
a length of between about 4 cm and about 15 cm;
a collapsed diameter of between about 10 and about 16 french;
a fully dilated diameter of between about 35 and about 55 mm;
wires of between about 0.008″ and about 0.018″ in diameter formed in a criss-cross pattern and defining spaces between the wires when the wire mesh body is its fully dilated diameter, wherein a majority of the spaces have an area between about 2 mm² and about 400 mm².

128-140. (canceled)

141. The assembly of claim 127 further comprising:
a catheter including:
a retractable catheter sheath;
a central tube positioned at least partially inside a retractable catheter sheath, the central tube passing through a center of the device; and
an outer tube positioned at least partially inside the retractable catheter sheath; and
a proximal retention end being affixed to the outer tube; and
a distal retention end being affixed to the central tube,
wherein the device is dilated by moving the distal retention end closer to the proximal retention end when the device is not retained in the retractable catheter sheath.

142. (canceled)
